(19) United States
(12) Patent Application Publication
(10) Pub. No.: US 2025/0077867 A1
(43) Pub. Date: Mar. 6, 2025

(54) AUTO-ENCODING DEVICE FOR SYNTHESIZABLE MOLECULE GENERATION MODEL BASED ON MOLECULAR STRUCTURE CONDITIONS AND MOLECULE GENERATION METHOD USING SAME

(71) Applicants: LG MANAGEMENT DEVELOPMENT INSTITUTE CO., LTD., Seoul (KR); KOREA ADVANCED INSTITUTE OF SCIENCE AND TECHNOLOGY, Daejeon (KR)

(72) Inventors: Kiyoung KIM, Seoul (KR); Honglak LEE, Seoul (KR); Sehui HAN, Seoul (KR); Moontae LEE, Seoul (KR); Daewoong JEONG, Seoul (KR); Yousung JUNG, Daejeon (KR); Juhwan NOH, Daejeon (KR)

(21) Appl. No.: 18/952,998

(22) Filed: Nov. 19, 2024

Related U.S. Application Data
(63) Continuation of application No. PCT/KR2023/005431, filed on Apr. 21, 2023.

(30) Foreign Application Priority Data
May 19, 2022 (KR) 10-2022-0061619
Jan. 10, 2023 (KR) 10-2023-0003635

(51) Int. Cl. G06N 3/08

(52) U.S. Cl. CPC G06N 3/08

(57) ABSTRACT

An autoencoding device for a synthesizable molecule generative model may include a memory configured to store chemical reaction data, chemical reaction learning information, and molecular structure information; and a processor configured to learn a neural network for a molecular generative model based on the chemical reaction data, the chemical reaction learning information, and the molecular structural information. The molecular generative model may include: a reaction sequence encoder configured to generate a latent space based on the chemical reaction learning information; a seed molecule encoder configured to generate an embedding space based on seed structural information for a seed molecule that is a final product of chemical reaction; and a decoder configured to output a reconstruction reaction sequence by decoding a reaction molecule predictive neural network and a reaction template prediction model generated from the latent space and the embedding space.

### CROSS REFERENCE TO RELATED APPLICATION(S)

This application is a continuation of International Application No. PCT/KR2023/005431, filed on Apr. 21, 2023, which claims the priority to Korean Patent Application No. 10-2022-0061619, filed on May 19, 2022, and Korean Patent Application No. 10-2023-0003635, filed on Jan. 10, 2023, which are all hereby incorporated by reference in their entireties.

### TECHNICAL FIELD

The present disclosure generally relates to an auto-encoding device for a molecular generative model and a molecule generation method. More specifically, some embodiments of the present disclosure relate to an auto-encoding device for a synthesizable molecule generative model considering molecular structural conditions and a method of generating a molecule using the same.

### BACKGROUND

Recently, various concepts and learning models are being developed in the field of artificial intelligence technology, and research on data prediction using these is actively being conducted. When predicting data based on artificial intelligence-based neural networks, learning or prediction algorithms for learning models are being developed to derive results with high prediction probability.

Molecular creation models are algorithms that have recently been gaining attention in the field of new material development because they may reverse engineer molecules with desired properties. However, existing molecular creation models may not consider the synthesizable nature of molecules. Therefore, a new methodology is required that may simultaneously generate chemical structures and synthetic routes with desired properties.

### SUMMARY

Some embodiments of the present disclosure may provide an auto-encoding device for a synthesizable molecule generative model considering molecular structural conditions and a molecule generation method using the same.

The problems to be solved by the present disclosure are not limited to the problems mentioned above, and other problems not mentioned will be clearly understood by those skilled in the art from the description below.

According to an exemplary embodiment of the present disclosure, an autoencoding device for a synthesizable molecule generative model may include a memory storing chemical reaction data, chemical reaction learning information, and molecular structure information; and a processor configured to learn a neural network that implements a molecular generative model based on the chemical reaction data, the chemical reaction learning information, and the structural information, wherein the molecular generative model may include: a reaction sequence encoder that generates a latent space based on the chemical reaction learning information; a seed molecule encoder that generates an embedding space based on seed structural information for the seed molecule that is a final product of the chemical reaction; and a decoder that outputs a reconstruction reaction sequence by decoding a reaction molecule predictive neural network and a reaction template prediction model generated from the latent space and the embedding space.

In addition, a method of generating a molecular model using a computing device according to an exemplary embodiment of the present disclosure for achieving the above-described technical problem may include obtaining a latent space comprising chemical reaction learning information; obtaining an embedding space comprising structural information but omitting synthetic path information of a molecule; sampling similar molecules from the latent space and the embedding space through a generative model, and generating candidate molecules; generating a synthetic pathway between molecules with seed structural information; and outputting a seed molecular model that comprises the seed structural information and the synthetic pathway.

According to certain embodiments of the present disclosure, by forming and implementing a chemical bonding space composed of synthesizable compounds, a chemical reaction database capable of generating compounds having high synthesizable properties and at the same time required properties may be constructed.

In addition, according to some embodiments of the present disclosure, the time for filtering out synthesizable molecules among new substances generated by a molecular generative model and deriving a synthetic path may be shortened through a variational auto-encoder-based machine learning methodology capable of simultaneously generating the chemical structure of a molecule having required properties and a chemical reaction path (synthetic path).

The effects of the present disclosure are not limited to the effects mentioned above, and other effects not mentioned will be clearly understood by those skilled in the art from the description below.

### BRIEF DESCRIPTION OF THE DRAWINGS

FIG. 1 is a schematic diagram schematically illustrating a molecular generative model according to an exemplary embodiment of the present disclosure.

FIG. 2 is a flow chart illustrating a method of operating an auto-encoding device for a synthetic molecule generative model according to an exemplary embodiment of the present disclosure.

FIG. 3 is a schematic diagram illustrating a process for generating chemical reaction data for a synthetic molecule generative model according to an exemplary embodiment of the present disclosure.

FIG. 4 is a flow chart illustrating a method for generating chemical reaction data for a synthetic molecule generative model according to an exemplary embodiment of the present disclosure.

FIG. 5 is a schematic diagram illustrating an auto-encoding device for a synthesizable molecule generative model according to an exemplary embodiment of the present disclosure.

FIG. 6 is a conceptual diagram for illustrating the operation of an auto-encoding device for a synthetic molecule generative model according to an exemplary embodiment of the present disclosure.

FIG. 7 is a conceptual diagram illustrating the operation of an auto-encoding device for a synthetic molecule generative model according to an exemplary embodiment of the present disclosure.

FIG. 8 is a flowchart illustrating a method of operating an auto-encoding device for a synthesizable molecule generative model according to an exemplary embodiment of the present disclosure.

FIG. 9 is a conceptual diagram illustrating the operation of an autoencoding device for a synthesizable molecule generative model according to an exemplary embodiment of the present disclosure.

FIG. 10 is a graph showing the performance of an autoencoding device for a synthesizable molecule generative model according to an exemplary embodiment of the present disclosure.

FIG. 11 is a graph showing the performance of an autoencoding device for a synthesizable molecule generative model according to an exemplary embodiment of the present disclosure.

FIG. 12 is a conceptual diagram illustrating the performance of an autoencoding device for a synthesizable molecule generative model according to an exemplary embodiment of the present disclosure.

### DETAILED DESCRIPTION OF EXEMPLARY EMBODIMENTS

Throughout the present disclosure, identical reference numerals refer to the same components. The present disclosure does not describe all elements of the embodiments, and general contents within the technical field to which the present disclosure or overlapping details between embodiments pertains are omitted. The terms “unit,” “module,” “member,” and “block” used in the specification may be implemented in either software or hardware, and according to embodiments, it is also possible for a plurality of “units,” “modules,” “members,” and “blocks” to be implemented as a single component, or for one “unit,” “module,” “member,” and “block” to include a plurality of components.

Throughout this specification, when a part is described as being “connected” to another part, this includes not only cases where they are directly connected, but also cases where they are indirectly connected, and an indirect connection includes a connection via a wireless communications network.

In addition, when a part is said to “include” a certain component, this means that it may further include other components rather than excluding other components, unless specifically stated to the contrary.

Throughout this specification, when a member is said to be located “on” another member, this includes not only the case where the member is in contact with the other member, but also the case where another member exists between the two members.

The terms such as first, second are used to distinguish one component from another component, and the components are not limited by the aforementioned terms.

Singular expressions include plural expressions unless the context clearly indicates otherwise.

The identification symbols in the each step are used for the convenience of explanation and do not explain the order of the each step, and each step may be performed in a different order than specified unless the context clearly indicates a specific order.

Hereinafter, the principles of operation and embodiments of the present disclosure will be described with reference to the accompanying drawings.

Before the explanation, the meaning of terms used in this disclosure will be briefly explained. However, since the explanation of terms is intended to help the understanding of this specification, it should be noted that they should not used in a meaning that limits the technical idea of this disclosure unless explicitly stated to limit the disclosure.

In this specification, the terms neural network, artificial neural network, and network function are often used interchangeably.

Also, throughout this specification, the terms neural network, neural network, and network function may be used interchangeably. A neural network may comprise a set of interconnected computational units, which may generally be referred to as “nodes.” These “nodes” may also be referred to as “neurons.” A neural network include at least two or more nodes. The nodes (or neurons) included in the neural networks may be interconnected by one or more “links.”

The present disclosure discloses a computing device or a computer as an example of an electronic processing device. For example, the computer device may be a server, a workstation, or the like, as an electronic processing device that processes information by performing communication with an external device, such as an application server, a computing server, a database server, a file server, a game server, a mail server, a proxy server, and a web server. Alternatively, the computing device may include a notebook, desktop, laptop, tablet personal computer (PC), slate PC, etc. equipped with a web browser.

A computing device may include one or more of a memory, a processor, an input unit, and/or an output unit. The artificial intelligence-related function according to some embodiments of the present disclosure may be operated through the processor and the memory.

The memory may store at least one instruction and various data required for artificial intelligence learning. According to one embodiment of the present disclosure, the memory may include various types of storage media including at least one of a flash memory type, a hard disk type, a multimedia card micro type, a card type memory (for example, an SD or XD memory, etc.), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, an optical disk, resistive memory cells such as ReRAM (resistive RAM), a PRAM (phase change RAM), an MRAM (magnetic RAM), an MRAM (Spin-Transfer Torgue MRAM), a CBRAM (conductive bridging RAM), and a FeRAM (ferroelectric RAM).

The processor may be configured to perform at least one instruction for artificial intelligence learning. The processor of certain embodiments of the present disclosure may be configured with one or more cores, and may include a processor for data analysis and deep learning, such as a neural processing unit (NPU), a central processing unit (CPU), a general purpose graphics processing unit (GPGPU), and a tensor processing unit (TPU) of a computing device. The processor may read a computer program stored in a memory and perform data processing for machine learning according to an embodiment of the present disclosure.

The processor may be a general-purpose processor such as a CPU, an AP, a DSP (Digital Signal Processor), a graphics-only processor such as a GPU, a VPU (Vision Processing Unit), or an AI-only processor such as an NPU. One or more processors are controlled to process input data according to predefined operation rules or AI models stored in a memory. Alternatively, when one or more processors are AI-only processors, the AI-only processor may be designed with a hardware structure specialized for processing a specific AI model.

According to one embodiment of the present disclosure, a processor may perform operations for learning a neural network. The processor may perform calculations for learning a neural network, such as processing input data for learning in deep learning (DL), extracting features from input data, calculating errors, and updating weights of a neural network using backpropagation. At least one of the NPU, CPU, GPGPU, and TPU of the processor may process learning of a network function. For example, the CPU and GPGPU may together process learning of a network function and classification of data using a network function. In addition, in one embodiment of the present disclosure, processors of a plurality of computing devices may be used together to process learning of a network function and classification of data using a network function. In addition, a computer program executed in a computing device according to one embodiment of the present disclosure may be a CPU, GPGPU, or TPU executable program.

The predefined operation rules or artificial intelligence models may be created through learning. Here, being created through learning may mean that a basic artificial intelligence model is learned by using a plurality of learning data by a learning algorithm, thereby creating predefined operation rules or an artificial intelligence model set to perform a desired characteristic (or purpose). Such learning may be performed in a device itself on which artificial intelligence according to an embodiment of the present disclosure is performed, or may be performed through a separate or external server and/or system. Examples of the learning algorithm include supervised learning, unsupervised learning, semi-supervised learning, or reinforcement learning, but are not limited to the examples described above.

The AI model may be a single AI model or may be implemented as multiple AI models. The AI model may be composed of a neural network (or an artificial neural network) and may include a statistical learning algorithm that mimics biological neurons in machine learning and cognitive science. The neural network may refer to a model in which artificial neurons (nodes) that form a network by combining synapses change the binding strength of synapses through learning, thereby having a problem-solving ability. The neurons of the neural network may include a combination of weights or biases. The neural network may include one or more layers composed of one or more neurons or nodes. For example, a device 100 may include an input layer, a hidden layer, and an output layer. The neural network constituting the device 100 may infer a desired result (output) from an arbitrary input (input) by changing the weights of neurons through learning.

The processor may create a neural network, train (or learn) a neural network, perform computations based on received input data, generate information signals based on the results of the computations, or retrain a neural network. Neural network models include CNN (Convolution Neural Network), R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), and RBM (Restrcted Boltzman Machine) such as GoogleNet, AlexNet, VGG Network, etc.), Fully Convolutional Network, LSTM (Long Short-Term Memory) Network, Classification Network, Generative Modeling, explainable AI, Continual AI, Representation Learning, AI for Material Design, BERT for natural language processing, SP-BERT, MRC/QA, Text Analysis, Dialog System, GPT-3, GPT-4, Visual Analytics for vision processing, Visual Understanding, Video Synthesis, Anomaly Detection for ResNet data intelligence, Time-Series Forecasting, Optimization, It may include various types of models such as Recommendation, Data Creation, etc., but not limited thereto. The processor may be implemented as one or more processors for performing operations according to models of the neural network. For example, the neural network may include a deep neural network.

Neural networks include CNN (Convolutional Neural Network), RNN (Recurrent Neural Network), perceptron, multilayer perceptron, FF (Feed Forward), RBF (Radial Basis Network), DFF (Deep Feed Forward), and LSTM. (Long Short Term Memory), GRU (Gated Recurrent Unit), AE (Auto Encoder), VAE (Variational Auto Encoder), DAE (Denoising Auto Encoder), SAE (Sparse Auto Encoder), MC (Markov Chain), HN (Hopfield) Network), BM (Boltzmann Machine), RBM (Restricted Boltzmann Machine), DBN (Depp Belief Network), DCN (Deep Convolutional Network), DN (Deconvolutional Network), DCIGN (Deep Convolutional Inverse Graphics Network), GAN (Generative Adversarial Network)), Liquid State Machine (LSM), Extreme Learning Machine (ELM), It may include ESN (Echo State Network), DRN (Deep Residual Network), DNC (Differentiable Neural Computer), NTM (Neural Turning Machine), CN (Capsule Network), KN (Kohonen Network) and AN (Attention Network). It will be appreciated by those skilled in the art that this may include any neural network, but is not limited thereto.

The network unit according to one embodiment of the present disclosure may configured to perform communication between a plurality of computing devices so that operations for determining a user control operation range or learning a model may be performed in a distributed manner on each of the plurality of computing devices. The network unit may enable communication between a plurality of computing devices so that operations for autism diagnosis or model learning using a network function may be processed in a distributed manner. The network unit according to one embodiment of the present disclosure may operate based on any form of wired and/or wireless communication technology implemented, such as short-distance (short-distance), long-distance, wired, and wireless, and may also be used in other networks.

An output unit according to one embodiment of the present disclosure may display a user interface (UI) for providing a result of determining a user control operation range and a judgment. The output unit may output any form of information generated, calculated, or determined by a processor and any form of information received by a network unit. In one embodiment of the present disclosure, the output unit may include at least one of a liquid crystal display (LCD), a thin film transistor-liquid crystal display (TFT LCD), an organic light-emitting diode (OLED), a flexible display, and/or a three-dimensional display (3D display). Some of these display modules may be configured as transparent or light-transmitting. This may be referred to as a transparent display module, and examples of the transparent display module include, but are not limited to, TOLED (Transparent OLED).

An input unit according to one embodiment of the present disclosure may receive a user input. The input unit may have keys and/or buttons on a user interface for receiving a user input, or physical keys and/or buttons. A computer program for controlling a display according to certain embodiments of the present disclosure may be executed according to a user input through the input unit.

The input unit according to some embodiments of the present disclosure may receive a signal by detecting a user's button operation or touch input, or may receive a user's voice or motion through a camera or microphone and convert it into an input signal. For this purpose, speech recognition technology or motion recognition technology may be used.

The input unit according to certain embodiments of the present disclosure may be implemented as an external input device connected to an external system. For example, the input device may be at least one of a touchpad, a touch pen, a keyboard, or a mouse for receiving user input, but this is only an example and is not limited thereto.

An input unit according to one embodiment of the present disclosure may recognize a user touch input. The input unit according to one embodiment of the present disclosure may have the same configuration as the output unit. The input unit may be configured with a touch screen implemented to receive a user's selection input. The touch screen may use any one of a contact-type electrostatic capacitance method, an infrared optical sensing method, a surface-acoustic (SAW) method, a piezoelectric method, and a resistive film method. The detailed description of the touch screen described above is only an example according to one embodiment of the present disclosure, and various touch screen panels may be employed in a computing device. The input unit configured with a touch screen may include a touch sensor. The touch sensor may be configured to convert a change in pressure applied to a specific portion of the input unit or electrostatic capacitance occurring in a specific portion of the input unit into an electrical input signal. The touch sensor may be configured to detect not only the position and area of the touch, but also the pressure at the time of the touch. When there is a touch input to the touch sensor, a corresponding signal(s) is sent to the touch controller. The touch controller may process the signal(s) and then transmit corresponding data to the processor, thereby enabling the processor to recognize which area of the input section has been touched, etc.

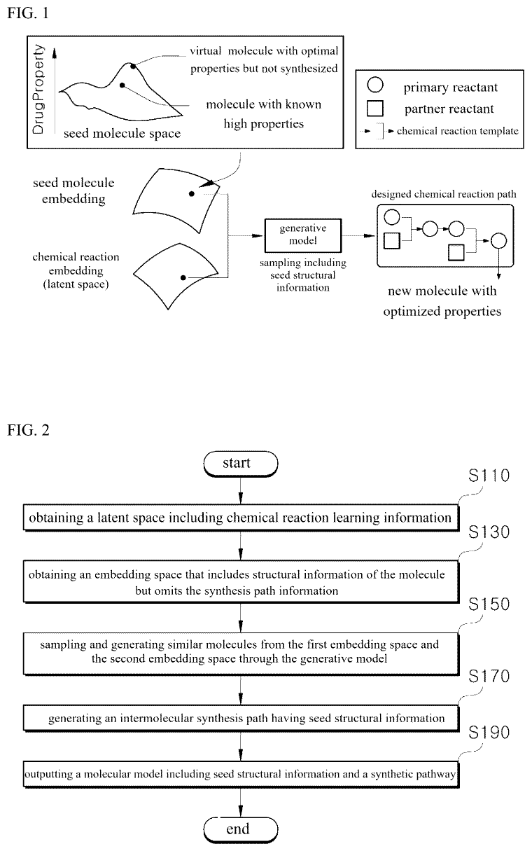

FIG. 1 is a schematic diagram schematically illustrating a molecular generative model according to an exemplary embodiment of the present disclosure. The molecular generative model according to some embodiments of the present disclosure may be implemented through the computing device described above.

AI and machine learning-based new material design methods are technologies that may accelerate material design in various fields such as drugs, OLEDs, and solar cells, and performance verification procedures are being carried out through the discovery and synthesis of new materials by applying machine learning methodologies in various fields. In particular, in the case of generative models, the machine learning methodology may be a methodology that may simulate the distribution of data, and be able to generate new data from the learned probability distribution. In the case of organic molecules, various types of molecular generative models have been proposed using string-based expressor or molecular graph expressor, such as SMILES (Simplified Molecular Input Line Entry System), and additionally, models that may generate molecules with desired properties by utilizing information on physical properties have been reported.

In the case of existing molecular generative models, there is no explicit information about the synthetic possibility directly related to experimental verification. Therefore, a method to extract the features of existing known molecules and obtain the degree of synthetic possibility or to reversely estimate the synthetic path of a given molecule may be needed.

Illustrative embodiments of the present disclosure present a methodology for designing directly synthesizable molecules via generative models, and specifically propose novel models in which information about chemical reaction pathways is learned in addition to information about individual molecular structures.

Referring to FIG. 1, the concept of a molecular generative model according to some embodiments of the present disclosure will be explained. The molecular generative model according to an exemplary embodiment of the present disclosure utilizes structural information of a molecule desired by a user (i.e., seed molecule embedding) as a condition along with a latent space (i.e., reaction embedding) in which information about a chemical reaction is learned, thereby maintaining (structure-preserving) chemical structural information of a specific molecule and generating a reaction path (designed reaction path) or a synthesis pathway of the molecule as a result of sampling.

In the case of generative models related to existing chemical reactions, since new chemical reactions are randomly obtained from the learned latent space, structural information specifically desired by the user is not reflected, and additional post-processing work is required for this, which has a disadvantage. In contrast, in the case of the molecular generative model according to some embodiments of the present disclosure, the structural information of molecules (synthetically unknown optimized molecules) that have optimized properties but whose synthetic paths are not known through the existing generative model are used as conditions to generate chemical reactions, so that the user may simultaneously generate synthetic paths while utilizing structural information specifically desired by the user.

According to certain embodiments of the present disclosure, by forming and implementing a chemical bonding space composed of synthesizable compounds, a chemical reaction database capable of generating compounds having high synthesizable properties and at the same time required properties may be constructed. In addition, according to certain embodiments of the present disclosure, the time for filtering out synthesizable molecules among the new substances generated by the molecular generative model and deriving the synthetic path may be shortened through a variational auto-encoder-based machine learning methodology that may simultaneously generate the chemical structure and chemical reaction path (synthetic path) of a molecule having the required properties.

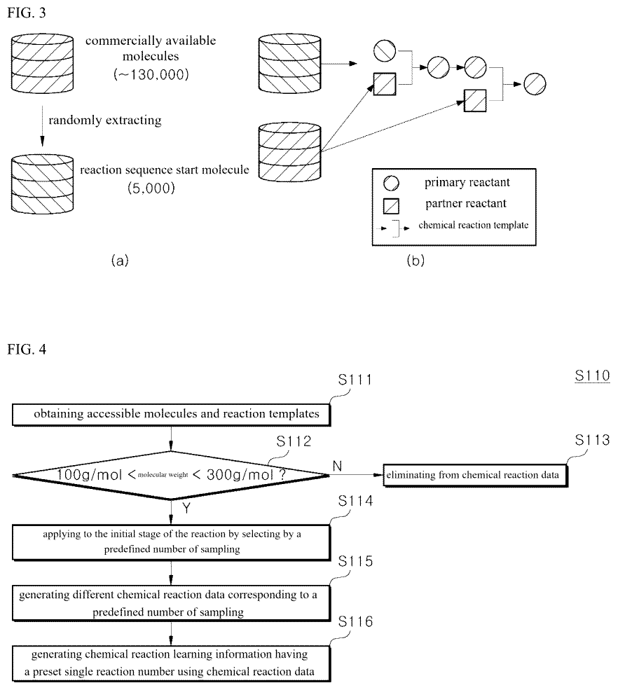

FIG. 2 is a flow chart illustrating a method of operating an auto-encoding device for a synthetic molecule generative model according to an exemplary embodiment of the present disclosure.

An auto-encoder may be a device configured to learn a neural network model configured to learn a method of compressing a given data distribution into a latent space, or apply and utilize a learned model. The autoencoder may include at least one encoder configured to compress data and at least one decoder configured to decompress data. In an exemplary embodiment, when input data is input to the encoder, the encoder may convert an image into a latent vector in the latent space, and the decoder may restore the latent vector back to the image to generate output data. In this case, the vector in the latent space that has undergone compression by the encoder is reduced in size compared to the input data, and the performance of the model may be known depending on how much the reduced input data is restored to the same size. The auto-encoder may be implemented by the computing device described above.

In step S110, the auto-encoding device may obtain a latent space including chemical reaction learning information. According to an exemplary embodiment of the present disclosure, the chemical reaction learning information may include information used for artificial intelligence learning as data on chemical reactions. The chemical reaction learning information may be commercially available chemical reaction data which may include generally known reaction templates. The auto-encoding device may convert the chemical reaction learning information into a latent space.

In step S130, the auto-encoding device may obtain an embedding space that includes structural information of the molecule without the synthesis pathway information. In the case of the molecule generative model according to an embodiment of the present disclosure, structural information of molecules that are designed through an existing generative model, have optimized properties and do not know the synthesis pathway may be utilized as a condition.

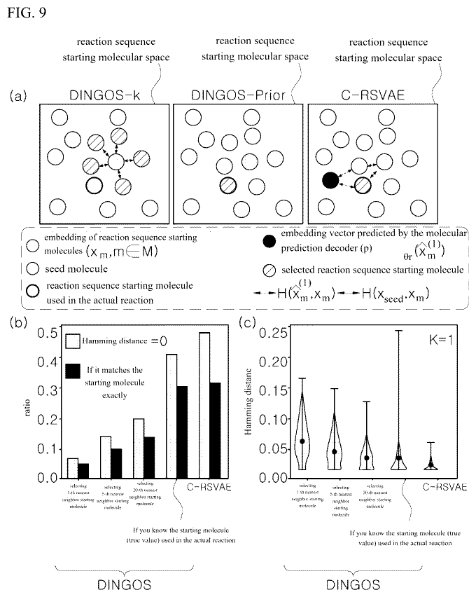

In step S150, the auto-encoding device may sample and generate similar molecules from the first embedding space and the second embedding space through the generative model. The similar molecule sampling may mean response sampling based on seed molecule conditions. Step S150 will be described in more detail with reference to FIG. 8.

In step S170, the auto-encoding device may generate an intermolecular synthesis pathway having seed structural information. In an embodiment of the present disclosure, the synthesis pathway may be interpreted in a similar meaning to an intermolecular reaction path. Step S170 may further include subroutines, a step of sampling similar molecules from a latent space and an embedding space through a generative model, a step of generating candidate molecules, and a step of generating an intermolecular synthesis pathway having seed structural information.

In step S190, the auto-encoding device may output a molecular model including seed structural information and a synthetic pathway. According to an embodiment of the present disclosure, by simultaneously generating a chemical structure of a molecule having required properties and a chemical reaction pathway (synthetic pathway), the time for filtering out synthesizable molecules among the new substances generated by the generative model and deriving a synthetic pathway may be shortened.

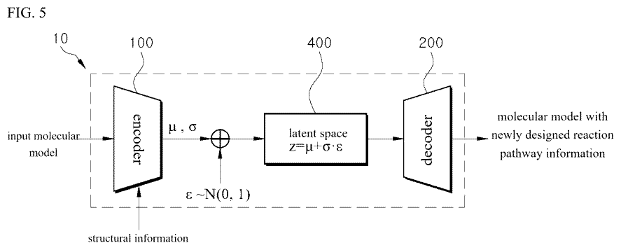

FIG. 3 is a schematic diagram illustrating a process for generating chemical reaction data for a synthetic molecule generative model according to an exemplary embodiment of the present disclosure.

According to an exemplary embodiment of the present disclosure illustrated in (a) of FIG. 3, chemical reaction data utilized for learning a molecular generative model may be constructed using about 150,000 molecules that are accessible or available for learning and 58 reaction templates that are mainly used for drug synthesis. In this example, among the total available molecules, about 130,000 molecules having a molecular weight of 100 to 300 g/mol (gram/mole) may be utilized, and 5,000 molecules may be randomly selected and utilized in the initial stage of the reaction.

In (b) of FIG. 3, molecules and reaction templates may be arbitrarily selected to generate reaction data. In an exemplary embodiment, a total of 2.1 million different chemical reaction data may be constructed, and each chemical reaction may be composed of up to three single reactions.

According to an exemplary embodiment of the present disclosure, the chemical reaction data includes a primary reactant and a partner reactant defined as a sequence, and the primary reactant and the partner reactant may be converted into a binary embedding vector.

According to an exemplary embodiment of the present disclosure, the chemical reaction data includes a reaction template defined as a sequence, and the reaction template may be converted into a one-hot vector.

FIG. 4 is a flowchart illustrating a method for generating chemical reaction data for a synthetic molecule generative model according to an exemplary embodiment of the present disclosure.

Step S110 of FIG. 2 of acquiring potential space may include steps S111 to S116.

In step S111, the auto-encoding device may obtain accessible or learnable molecules and reaction templates. For example, according to an exemplary embodiment of the present disclosure, chemical reaction data utilized for learning a molecular generative model may be constructed using about 150,000 molecules that are previously accessible or available for learning and 58 reaction templates that are mainly utilized for drug synthesis. In this case, among the total available molecules, about 130,000 molecules having a molecular weight of 100 to 300 g/mol (gram/mole) may be utilized.

In step S112, the auto-encoding device may determine whether the molecular weight is within a predefined range. In the method for generating a molecular model using a computing device according to an exemplary embodiment of the present disclosure, the predefined range may be greater than 100 g/mol and less than 300 g/mol.

In step S113, the auto-encoding device may remove molecules having molecular weights out of the predefined range from the chemical reaction data. For example, the auto-encoding device may not store molecules having molecular weights smaller than 100 g/mol or larger than 300 g/mol in the chemical reaction data, or may remove the corresponding molecule data from the already stored chemical reaction data.

In step S114, the auto-encoding device may be applied to the initial stage of the reaction by selecting a predefined number of samples. For example, the auto-encoding device may be utilized in the initial stage of the reaction by selecting a number of sample molecules of 5,000. In step S115, the auto-encoding device may generate different chemical reaction data corresponding to the predefined number of samples.

In step S116, the auto-encoding device may generate chemical reaction learning information having a preset single reaction number using chemical reaction data. In an exemplary embodiment of the present disclosure, the preset single reaction number may be 3.

FIG. 5 is a schematic diagram illustrating an auto-encoding device for generating a synthesizable molecular model according to an exemplary embodiment of the present disclosure.

An auto-encoding device 10 may include at least one encoder 100 for compressing data and at least one decoder 200 for decompressing data. In an exemplary embodiment, input data (e.g., a molecular model (MM)) may be input to the encoder 100, and structural information (SI) of a molecule may be further input as a condition to the encoder 100. In an exemplary embodiment, the encoder 100 may convert an image into a latent vector (z) on a latent space 400, and the decoder 200 may restore the latent vector (z) to generate output data (e.g. a decoded molecular model (dMM)). Here, the output data may include a reaction path.

The auto-encoding device 10 according to an embodiment of the present disclosure may include an auto-encoder configured to arrange and place data on the latent space 400 according to a predefined distribution. The auto-encoding device 10 according to an exemplary embodiment may arrange and place data according to a normalized Gaussian distribution. According to an exemplary embodiment, the auto-encoding device 10 may comprise an auto-encoder configured to arrange and place learning data having similar characteristics on a latent space so that the learning data are not arranged in a scattered manner on the latent space 400.

According to an exemplary embodiment of the present disclosure, the encoder 100 may transform input data (MM) into a latent vector (z) on the latent space 400. The input data (MM) may be classified to follow a normal distribution having a mean (μ) and a standard deviation (σ).

After the encoding is performed, the latent vector (z) may be added with noise (ε) that has the characteristic of following a standard normal distribution (i.e., a normal distribution with a mean of 0 and a standard deviation of 1).

In this case, the latent vector (z) may follow Formula 1.

$$z = μ + σ \cdot ε \qquad [ Formula 1 ]$$

where z represents a latent vector, u represent a mean, ø represents a standard deviation, and ¿ represents noise.

Referring to Formula 1, noise (¿) may function as a weight of variance.

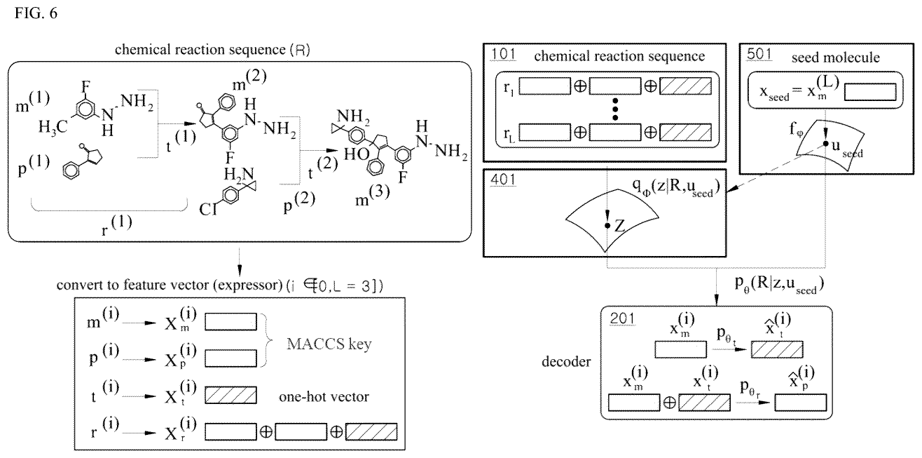

FIG. 6 is a conceptual diagram illustrating the operation of an auto-encoding device for a synthetic molecule generative model according to an exemplary embodiment of the present disclosure. A generative model configured to generate a reaction path by utilizing information on a target molecular structure as a condition according to an embodiment of the present disclosure is illustrated in FIG. 6. A molecular structure condition-based synthetic molecule generative model according to an embodiment of the present disclosure may be used interchangeably with a C-RSVAE (Conditional-Reaction Sequence Variational Autoencoder).

An auto-encoding device for a synthesizable molecule generative model according to an exemplary embodiment of the present disclosure may include a memory for storing chemical reaction data, chemical reaction learning information, and structural information of a molecule, and a processor configured to learn an artificial neural network for implementing a molecule generative model based on the chemical reaction data, chemical reaction learning information, and structural information.

In an embodiment of the present disclosure, chemical reaction data may be defined in the form of a sequence. Therefore, a chemical reaction including n single reactions may be defined as a reaction sequence (R) as expressed in Formular 2.

$$R = [ {r}^{( 0 )} , {r}^{( 1 )} , … , {r}^{( n )} , {r}^{( n + 1 = L )} ] \qquad [ Formula 2 ]$$

Here, r⁽ⁱ⁾ represents the i-th single reaction, i=0 represents the starting state of the reaction, and i=n+1=L represents the final state of the reaction.

Additionally, in an embodiment of the present disclosure, the primary reactant may be defined as m⁽ⁱ⁾, the partner reactant may be defined as P⁽ⁱ), and the reaction template may be defined as t⁽ⁱ⁾.

Both the primary reactant (m⁽ⁱ⁾) and the partner reactant (P⁽ⁱ⁾) may be represented by a binary embedding vector called MACCS key.

When expressed as an embedding vector, the primary reactant (m⁽ⁱ⁾) vector may be expressed as xₘ⁽ⁱ⁾, the partner reactant (P⁽ⁱ⁾) vector may be expressed as xₚ⁽ⁱ⁾, and the reaction template (t⁽ⁱ⁾ may be expressed as a one-hot vector (xₜ⁽ⁱ⁾).

Through this, r⁽ⁱ⁾ may be expressed as xᵣ⁽ⁱ⁾, and may be obtained by concatenating

$${x}_{m}^{( i )} , {x}_{p}^{( i )} , {x}_{t}^{( i )} ( i . e . , {x}_{r}^{( i )} = {X}_{m}^{( i )} ⊕ {X}_{p}^{( i )} ⊕ {X}_{t}^{( i )}$$

In an exemplary embodiment, the above expression may be defined as Xₘ⁽⁰⁾=0, Xₚ⁽⁰⁾=Xₘ⁽¹⁾ for i=0, where Xₜ⁽⁰⁾ may be defined as a start token.

In an exemplary embodiment, the above expression may be defined as Xₚ^{(L)}=0 if i=L, and Xₜ^{(L)} may be defined as an end token.

In an embodiment of the present disclosure, a chemical reaction may be used as an input value of a generative model. In the learning process, the final product of a given chemical reaction may be defined as a seed molecule (seed). In contrast, an arbitrary molecule may be utilized in the testing process.

The molecular generative model may include a reaction sequence encoder 101 (q_{Φ}(z|R, U_{seed})) configured to generate a latent space 401 based on chemical reaction learning information, a seed molecule encoder (f_{φ}(X_{seed})) configured to generate an embedding space 501 based on seed structural information for a seed molecule, which is a final product of a chemical reaction, and a decoder 201 (p_{θ}(R|z, U_{seed})) configured to output a reconstructed reaction sequence by decoding a reaction molecule prediction neural network and a reaction template prediction model generated from the latent space 401 and the embedding space 501.

Here, φ, θ, Φ are all model parameters that are optimized through learning. And F (•) is defined as an arbitrary multilayer neural network.

f_{φ} is composed of a neural network and calculates the seed molecule embedding (U_{seed}) (i.e., u_{seed}=f_{φ}(x_{seed})), and the calculated U_{seed} is used as a condition when calculating the latent vector by encoding the chemical reaction.

The reaction sequence encoder 101 (q_{Φ}(z|R, u_{seed})) encodes the calculated seed molecule embedding (U_{seed}) and the chemical reaction (R) to calculate the latent vector (Z), and recognizes the chemical reaction steps as data in the form of a sequence to calculate the embedding vector for the entire reaction through a bidirectional gated recurrent unit, which is a type of RNN (Recurrent Neural Network).

In this case, according to the definition of variational autoencoder (VAE), the latent vector (Z) is modeled as a normal distribution, and the mean (u) and standard deviation (σ) are calculated using Formula 3.

$$⌈ μ , σ ⌉ = {F}_{h} ( BiGRU ( [ {x}_{r}^{( 1 )} , {x}_{r}^{( 2 )} , … , {x}_{r}^{( L )} ] \vert {u}_{seed} ) ) \qquad [ Formula 3 ]$$

Bidirectional GRU (BiGRU) is a model that recognizes a given sequence in both directions and calculates an embedding vector, and Fₕ is a neural network that calculates the mean (μ) and standard deviation (σ) using the previously calculated embedding vector.

According to an exemplary embodiment of the present disclosure, the reaction sequence encoder 101 may use an embedding vector as a condition when calculating the latent vector on the latent space 401.

According to an exemplary embodiment of the present disclosure, the reaction sequence encoder 101 may generate a latent vector as a result of encoding the chemical reaction of the embedding vector and the seed molecule.

According to an exemplary embodiment of the present disclosure, a reaction sequence encoder 101 may generate a latent embedding vector for an entire chemical reaction by learning chemical reaction data defined as a sequence based on a recurrent neural network.

According to an exemplary embodiment of the present disclosure, the reactive sequence encoder 101 may generate a latent embedding vector using a bidirectional gated recurrent unit, which is a recurrent neural network that recognizes a given sequence in both directions and is designed to include a forget gate.

According to an exemplary embodiment of the present disclosure, a seed molecule encoder (f_{φ}(x_{seed})) may provide an embedding vector encoding a chemical reaction of a seed molecule to a reaction sequence encoder 101.

The decoder 201 (p_{φ}(R|z, U_{seed})) may be composed of two types of neural networks: a response template prediction neural network (Pθₜ) and a reaction-composing molecule prediction neural network (Pθᵣ). Here, θₜ, and θᵣ are model parameters obtained through learning.

A reaction template prediction neural network (Pθₜ) may predict a template (xₜ⁽ⁱ⁾) for a given primary reactant (xₘ⁽ⁱ⁾).

A molecular prediction neural network (Per) predicts the primary reactant (xₘ⁽¹⁾=xₚ⁽⁰⁾ used in the initial step of a reaction and the partner reactant (xₚ^{(i<0)}) for a given primary reactant (xₘ^{(i>0)}) and reaction template (xₚ^{(i>0)}).

That is, the predicted template (\hat{X}ₜ^{(j)}) and reactant (\hat{X}ₚ^{(k)}) may be calculated using Formula 4 and Formula 5.

$${\hat{x}}_{p}^{( j )} ∼ {p}_{{θ}_{t}} ( {m}^{( j )} \vert z , {u}_{seed} ) = Softmax [ {F}_{t} ( GRU ( {x}_{m}^{( j )} \vert z , {u}_{seed} ) ) ] \qquad [ Formula 4 ]$$

$${\hat{x}}_{p}^{( j )} ∼ {p}_{{θ}_{r}} ( {m}^{( k )} , {t}^{( k )} \vert z , {u}_{seed} ) = Sigmoid [ {F}_{p} ( GRU ( {x}_{m}^{( k )} ⊕ {x}_{t}^{( k )} \vert z , {u}_{seed} ) ) ] \qquad [ Formula 5 ]$$

Here, j and k are integers satisfying j∈[1, L] and k∈[0, L−1], respectively, and Ft and Fₚ are neural network models that receive the output values of the GRU obtained in the 1st or k-th step as input values, respectively.

In an embodiment of the present disclosure, when generating a new molecule using a learned artificial neural network, an encoder may be omitted and only the learned decoder 201 (P_{θ}(R|z, u_{seed})) 201 may be utilized.

According to an exemplary embodiment of the present disclosure, the decoder 201 may include a reaction template prediction neural network, and a reaction-constituting molecule prediction neural network.

According to an exemplary embodiment of the present disclosure, a reaction template prediction neural network may be designed to predict a reaction template for a primary reactant that is a prediction target.

According to an exemplary embodiment of the present disclosure, a molecular prediction neural network may be designed to predict a primary reactant as a prediction target, a partner reactant for a reaction template, and a starting primary reactant utilized in a reaction initiation step.

According to an exemplary embodiment of the present disclosure, a latent vector may be modeled according to a normal distribution having a mean and a standard deviation, and the mean and the standard deviation may be calculated through a latent embedding vector.

According to an exemplary embodiment of the present disclosure, each of a synthetic pathway and a molecular structure may be mapped onto a latent space, and a molecule may be generated by reconstructing the synthetic pathway from latent vectors on the two latent spaces.

According to an exemplary embodiment of the present disclosure, since the decoder 201 sequentially creates a synthesis pathway to generate a molecule, all molecules generated may have a synthesis pathway.

Therefore, the auto-encoding device according to an exemplary embodiment of the present disclosure may generate a molecule similar to the seed molecule and having a synthetic path by utilizing the generative model when the synthesis of the seed molecule is difficult.

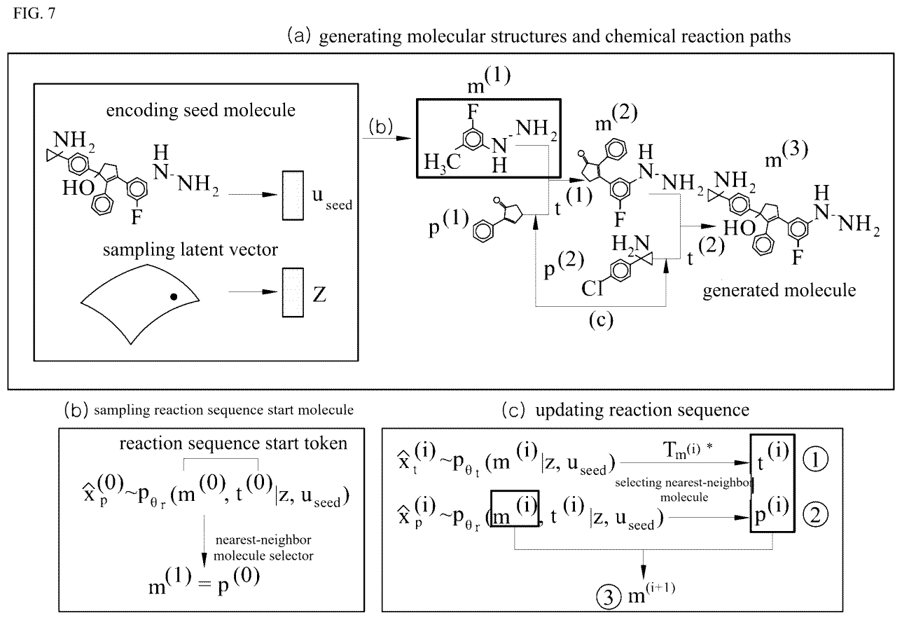

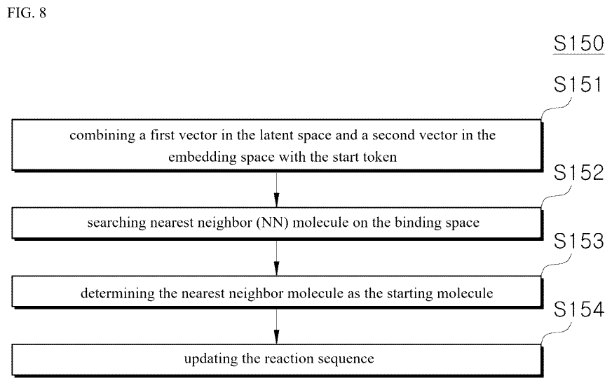

FIG. 7 is a conceptual diagram illustrating the operation of an auto-encoding device for a synthetic molecule generative model according to an exemplary embodiment of the present disclosure.

Referring to FIG. 7, sampling of new synthesizable molecules may be performed by utilizing the embedding vector (U_{seed}) for the seed molecule (seed) and the latent vector (Z) sampled from the latent space modeled as a normal distribution, and may be separated into the process of sampling the starting molecule ((b) of FIG. 7) and updating the reaction sequence ((c) of FIG. 7).

According to an embodiment of the present disclosure, since the reaction is formed with a molecule that is already learned or available, sampling may be performed based on the distance between the predicted result and the available molecule through the decoder. Te starting molecule (m⁽¹⁾) is defined using Formula 6.

$$[ {m}_{l}^{( 1 )} , … , {m}_{k}^{( 1 )} ] ∼ {kNN}_{m ∈ M} [ H ( {\hat{x}}_{m}^{( 1 )} , {x}_{m} ) + H ( {x}_{seed} , {x}_{m} ) ] \qquad [ Formula 6 ]$$

In this case, \hat{X}ₘ⁽¹⁾˜(m⁽⁰⁾, t⁽⁰⁾|z, u_{seed}) is the predicted result through the decoder, and kNNₘ may represent the k-nearest-neighbor sampling for the set (M) including the predicted result (\hat{X}ₘ⁽¹⁾) and the starting molecules defined in advance. The Hamming distance (H) may be used as a criterion for defining the adjacent neighbors. In this case, rather than simply utilizing the first Hamming distance (H(\hat{X}ₘ⁽¹⁾, Xₘ)) with the predefined molecule, the second Hamming distance (H(X_{seed}, Xₘ)) with the actual seed molecule may be additionally considered.

Referring to (c) of FIG. 7, the process of updating the reaction sequence comprising a step of predicting a reaction template (t⁽ⁱ⁾ and a step of predicting a partner reactant (P⁽ⁱ), and may be expressed using Formula 7 and Formula 8.

$${t}^{( i )} ∼ {T}_{m} ( i ) * {\hat{x}}_{t}^{( i )} \qquad [ Formula 7 ]$$

$$[ {p}_{l}^{( i )} , … , {p}_{k}^{( i )} ] ∼ {kNN}_{p ∈ P [ {t}^{( i )} ]} [ H ( {\hat{x}}_{p}^{( i )} , {x}_{p} ) ] \qquad [ Formula 8 ]$$

wherein, \hat{X}ₜ(i)˜p_{θ}t(m⁽ⁱ⁾|z, u_{seed}), \hat{X}ₚ⁽ⁱ⁾˜pθr (m⁽ⁱ⁾, t⁽ⁱ⁾|z, u_{seed}).

In an exemplary embodiment, when predicting a reaction template (t⁽ⁱ⁾), sampling is performed only for reactions applicable to a given primary reactant (m⁽ⁱ⁾), and for this purpose, a mask vector defined as Tm⁽ⁱ⁾ may be used.

In an exemplary embodiment, when predicting a partner reactant (P⁽ⁱ⁾), it may be inferred through k-nearest-neighbor sampling (kNN_{P[t}_{⁽ⁱ⁾}]) between the predicted result (\hat{X}ₚ⁽ⁱ⁾) and the set (P[t⁽ⁱ)]) of molecules applicable to a specific reaction template (t⁽ⁱ⁾), similar to the case of sampling the previous starting molecule, via the decoder. This reaction sequence update may be repeated until the predicted reaction template is an ‘end token’ or the maximum number of reaction steps (=3) is reached.

According to an embodiment of the present disclosure, an autoencoding device may generate a chemical reaction through sampling of seed molecules and reaction latent spaces by utilizing a learned generative model.

FIG. 8 is a flowchart illustrating a method of operating an auto-encoding device for a synthetic molecule generative model according to an exemplary embodiment of the present disclosure. Any description of the operation of FIG. 8 that overlaps with that of FIG. 7 will be omitted.

In step S151, the auto-encoding device may combine a first vector in the latent space and a second vector in the embedding space with the start token.

In step S152, the nearest neighbor (NN) molecule may be searched from the binding space. According to an exemplary embodiment of the present disclosure, the auto-encoding device may calculate a first Hamming distance between the predicted molecule and the predefined starting molecule through the decoder based on the Hamming distance. According to an exemplary embodiment of the present disclosure, a second Hamming distance between the starting molecule and the seed molecule may be calculated.

In an exemplary embodiment, the Hamming distance (H) is an indicator of the similarity of molecules. For instance, the smaller the Hamming distance, the more similar the properties of molecules may be.

In step S153, the auto-encoding device may determine the nearest neighbor molecule as the starting molecule of the chemical reaction. In step S154, the auto-encoding device may update the reaction sequence.

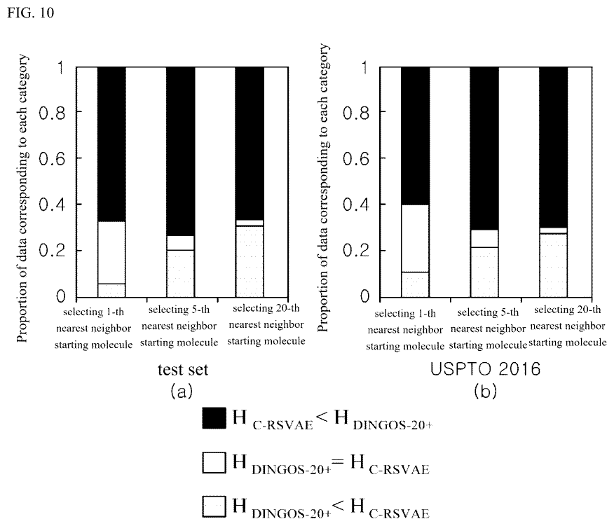

FIG. 9 is a conceptual diagram illustrating the operation of an auto-encoding device for a synthetic molecule generative model according to an exemplary embodiment of the present disclosure.

Referring to FIG. 9, the performance resulting from the application of the learned molecular generative model is explained.

(a) of FIG. 9 is a conceptual diagram of a model used to compare restoration performance.

In order to verify the in-domain seed molecule restoration performance of the model of an embodiment of the present disclosure, a comparison was performed with DINGOS, a methodology for generating chemical reactions capable of generating molecules similar to seed molecules through conventional machine learning methods and heuristic rule-based methods. In an exemplary embodiment, the in-domain seed molecules were randomly extracted from 1,000 data that were not used for learning among chemical reaction data (reaction database), and the restoration performance was compared.

A comparison group based on a total of four existing models (DINGOS-1, DINGOS-5, DINGOS-20, DINGOS-Prior) was set up. The DINGOS-k (k=1,5,20) method means selecting k molecules most similar to the seed molecule in the process of selecting the starting molecule in the DINGOS model, and in the case of DINGOS-Prior, since the chemical reaction information for the seed molecule is already known, the starting molecule corresponding to the actual true value is selected to form the chemical reaction.

The evaluation of the restoration performance was performed by utilizing the Hamming distance between the most similar molecule among the molecules obtained from each method and the seed molecule, and the ratio of cases where the value is 0 (cases where the same substructure information is present) and cases where the molecule is restored to be completely identical was calculated. In this case, when both the encoder and decoder according to an embodiment of the present disclosure were utilized, a restoration rate of 98% was achieved for 1,000 data.

(b) of FIG. 9 shows the ratio of molecules generated by the DINGOS method in which the Hamming distance to the seed molecule is 0 or is completely identical to the existing seed molecule.

As may be seen in (b) of FIG. B, in the case in which the DINGOS-k based method was used, as k increased, more similar molecules were generated. However, in the case in which DINGOS-Prior was used, the ratio increased by 2 to 7 times. This may mean that utilizing only the similarity with the existing seed molecule is insufficient for actually generating molecules similar to the seed molecule.

(c) of FIG. 9 shows the distribution of Hamming distances for the seed molecules of the generated molecules. Even when examining the distribution of Hamming distances between the actually generated molecules and the seed molecules, molecules more similar to the seed molecule are obtained in DINGOS-Prior.

Therefore, the molecule generation method (C-RSVAE) according to an embodiment of the present disclosure outperforms the DINGOS-k based method, and shows similar performance to DINGOS-Prior even though it does not provide information on the chemical reaction for the actual test molecule. In other words, by utilizing the latent space learned through an embodiment of the present disclosure, it is possible to generate molecules similar to the seed molecules of the existing in-domain region more effectively.

FIG. 10 is a graph showing the performance of an auto-encoding device for a synthesizable molecule generative model according to an exemplary embodiment of the present disclosure.

Referring to FIG. 10, the Hamming distance criterion performance between the generated molecule and the seed molecule is compared between the compound reactive volatilization (CRSVAE) methodology and the existing methods (e.g., DINGOS).

The main application of the methodology (C-RSVAE) implemented according to an embodiment of the present disclosure is the generation of new synthesizable molecules having structural information similar to that of the seed molecule, so it is necessary to evaluate the performance when seed molecules existing in the out-of-domain as well as in the actual in-domain are given as conditions. In the case of the in-domain, 1,000 new seed molecules that were not used for learning were sampled similarly to FIG. 9, and in the case of the out-of-domain, 1,000 seed molecules were randomly sampled and used among the molecules registered in 2016 at the United State Patent & Trademark Office (USPTO), which has reaction information on molecules registered as patents until 2016 (i.e., “USPTO 2016”). In this case, DINGOS-20+ was used as the reference model, which is a method of additionally sampling starting molecules when the number of sampled reactions is less than 300 in the case of DINGOS-20.

Among the molecules generated for performance comparison, the Hamming distance to the seed molecule was calculated, and the average value of the closest K (=1, 5, 20) molecules was calculated for each method of DINGOS-20+ and C-RSVAE (i.e., HDINGOS-20+, HC-RSVAE). Then, the magnitude of each calculated value was classified into three categories and compared and displayed. (e.g., HC-RSVAE<H_{DINGOS-20}+ (black), H_{DINGOS-20+}=H_{C-RSVAE} (white), H_{DINGOS-20+}<H_{C-RSVAE} (dot pattern)).

As may be seen in FIG. 10, when utilizing the methodology (C-RSVAE) proposed in this disclosure, the rate of generating molecules more similar to the seed molecule is high, and this proves that it is efficient to learn structural information and reaction information together.

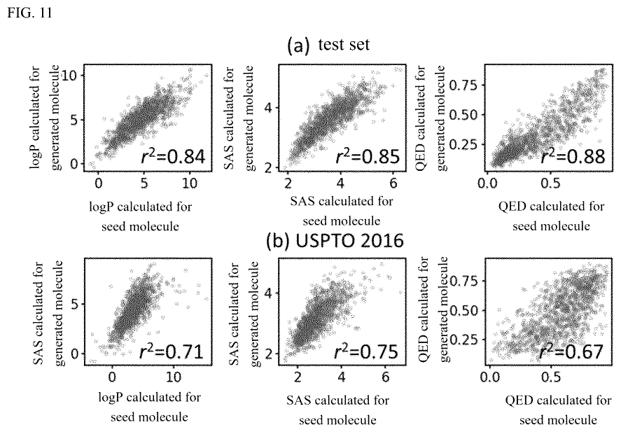

FIG. 11 is a graph showing the performance of an autoencoding device for a synthesizable molecule generative model according to an exemplary embodiment of the present disclosure.

Referring to FIG. 11, the correlation between the molecular properties (logP, SAS, QED) generated by the model implemented according to an embodiment of the present disclosure and the seed molecule is shown.

Based on the previous results, three properties of logP, SAS, and QED were calculated for the molecules generated through the model implemented according to an embodiment of the present disclosure, and the correlation with the seed molecule was compared. As may be seen in FIG. 11, both the test data and USPTO 2016 showed a high correlation. This relationship is consistent with the structure-property relationship that has been mainly accepted in the past.

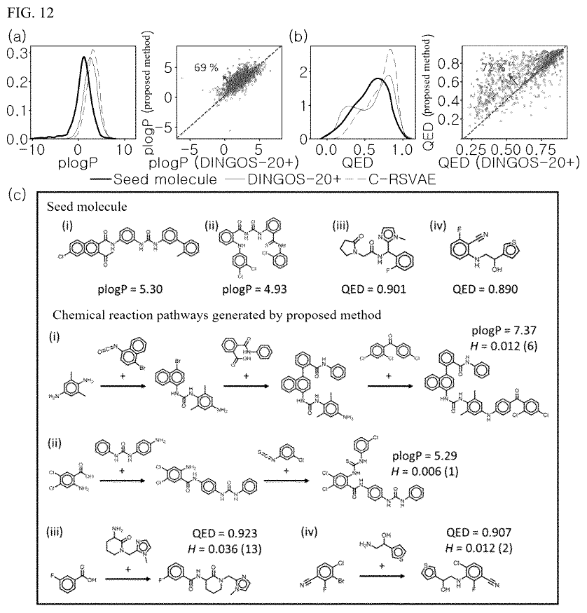

FIG. 12 is a conceptual diagram illustrating the performance of an auto-encoding device for a synthesizable molecule generative model according to an exemplary embodiment of the present disclosure.

(a) and (b) of FIG. 12 are graphs for comparing the distribution of properties (plogP, QED) of generated molecules and seed molecules, and (c) of FIG. 12 is a conceptual diagram showing the results of molecule generation that may reveal a new synthetic route based on a virtual molecule with optimized properties obtained through an existing generative model.

As may be seen in (a) and (b) of FIG. 12, most of the generated molecules may have improved property values (plogP, QED) than the seed molecule. This may show that the method implemented according to an embodiment of the present disclosure may obtain molecules with improved property values because random reactions are obtained from a given seed molecule and a randomly sampled latent vector. In addition, through comparison with DINGOS-20+ and the method implemented according to an embodiment of the present disclosure, the method implemented according to an embodiment of the present disclosure shows a higher rate of obtaining molecules with improved properties, and this may be interpreted as the increase in sampling diversity due to random sampling in the latent space where chemical reactions are learned.

In an exemplary embodiment, if the proposed model is used to generate a new synthesizable molecule based on a virtual molecule with existing optimized properties, a new molecule having a synthetic route with improved property values than the existing optimized molecule may be obtained for each of plogP and QED.

The method according to some embodiments of the present disclosure described above may be implemented as a program (or application) to be executed by being combined with a hardware server and stored on a medium. The disclosed embodiments may be implemented in the form of a recording medium storing instructions executable by a computer. The instructions may be stored in the form of program codes, and when executed by a processor, may generate a program module to perform the operations of the disclosed embodiments. The recording medium may be implemented as a computer-readable recording medium.

A computer-readable recording medium includes all types of recording media storing instructions that may be deciphered by a computer. In an exemplary embodiment, the recording medium may store a computer program in the form of a computer program code or an executable file. The program may include codes coded in a computer language, such as C, C++, JAVA, or machine language, that may be read by the processor (CPU) of the computer through the device interface of the computer so that the computer reads the program and executes the methods implemented as the program. Such codes may include functional codes related to functions that define functions necessary for executing the methods, and may include control codes related to execution procedures necessary for the processor of the computer to execute the functions according to a predetermined procedure. In addition, such codes may further include memory reference-related codes regarding which location (address address) of the internal or external memory of the computer should be referenced for additional information or media necessary for the processor of the computer to execute the functions. In addition, if the processor of the computer needs to communicate with another computer or server located remotely in order to execute the above functions, the code may further include communication-related code regarding how to communicate with another computer or server located remotely using the communication module of the computer, and what information or media to send and receive during communication.

The above-mentioned storage medium means a medium that stores data semi-permanently and may be read by a device, rather than a medium that stores data for a short period of time, such as a register, cache, or memory. Specifically, examples of the above-mentioned storage medium include, but are not limited to, ROM, RAM, CD-ROM, magnetic tape, floppy disk, or optical data storage device. That is, the above-mentioned program may be stored in various storage media on various servers that the computer may access or in various storage media on the user's computer. In addition, the above-mentioned medium may be distributed to computer systems connected to a network, so that a computer-readable code may be stored in a distributed manner.

The steps of a method or algorithm described in connection with the embodiments of the present disclosure may be implemented directly in hardware, implemented in a software module executed by hardware, or implemented by a combination of these. The software module may reside in a Random Access Memory (RAM), a Read Only Memory (ROM), an Erasable Programmable ROM (EPROM), an Electrically Erasable Programmable ROM (EEPROM), a flash memory, a hard disk, a removable disk, a CD-ROM, or any other form of computer-readable recording medium well known in the art to which the present disclosure pertains.

As described above, the disclosed embodiments have been described with reference to the attached drawings. Those skilled in the art to which the present disclosure pertains will understand that the present disclosure may be implemented in forms other than the disclosed embodiments without changing the technical idea or essential features of the present disclosure. The disclosed embodiments are exemplary and should not be construed as limiting.

What is claimed is:

1. An auto-encoding device comprising:
a memory configured to store chemical reaction data, chemical reaction learning information, and molecular structure information; and
a processor configured to learn a neural network for a molecular generative model based on the chemical reaction data, the chemical reaction learning information, and the molecular structural information, wherein the molecular generative model comprises:
a reaction sequence encoder configured to generate a latent space based on the chemical reaction learning information;
a seed molecule encoder configured to generate an embedding space based on seed structural information for a seed molecule that is a final product of chemical reaction; and
a decoder configured to output a reconstruction reaction sequence by decoding a reaction molecule predictive neural network and a reaction template prediction model generated from the latent space and the embedding space.

2. The autoencoding device of claim 1, wherein the seed molecule encoder is configured to provide an embedding vector encoding chemical reaction of the seed molecule to the reaction sequence encoder.

3. The autoencoding device of claim 2, wherein the reaction sequence encoder is configured to use the embedding vector when calculating a latent vector in the latent space.

4. The autoencoding device of claim 2, wherein the reaction sequence encoder is configured to generate a latent vector by encoding the embedding vector and the chemical reaction of the seed molecule.

5. The autoencoding device of claim 4, wherein the reaction sequence encoder is configured to generate a latent embedding vector for the chemical reaction by learning the chemical reaction data using a recurrent neural network.

6. The autoencoding device of claim 5, wherein the reaction sequence encoder is configured to generate the latent embedding vector using a bidirectional gated recurrent unit, a model designed to recognize a sequence bidirectionally and comprise a forget gate as the recurrent neural network.

7. The autoencoding device of claim 5, wherein the latent vector is modeled according to a normal distribution with a mean and standard deviation.

8. The autoencoding device of claim 7, wherein the mean and the standard deviation is calculated based on the latent embedding vector.

9. The autoencoding device of claim 1, wherein the decoder comprises a reaction template predictive neural network and a molecular predictive neural network for the chemical reaction.

10. The autoencoding device of claim 9, wherein the reaction template predictive neural network is configured to predict a reaction template for a primary reactant.

11. The autoencoding device of claim 9, wherein the molecular predictive neural network is configured to predict a partner reactant for a primary reactant and a reaction template, and an initial primary reactant utilized in a reaction initiation step.

12. The autoencoding device of claim 1, wherein:
the chemical reaction data comprises a primary reactant and a partner reactant, and
the primary reactant and the partner reactant are converted into binary embedding vectors.

13. The autoencoding device of claim 1, wherein:
the chemical reaction data comprises a reaction template, and
the reaction template is converted into a one-hot vector.

14. A computer-implemented method comprising:
obtaining a latent space comprising chemical reaction learning information;
obtaining an embedding space comprising structural information without synthetic path information of a molecule;
sampling similar molecules from the latent space and the embedding space using a generative model, and generating candidate molecules;
generating a synthesis pathway having seed structural information; and
outputting a seed molecular model that comprises the seed structural information and the synthesis pathway.

15. The method of claim 14, wherein:
the obtaining of the latent space comprises:
obtaining a learnable molecule and a reaction template;
determining whether a molecular weight of molecules included in chemical reaction data is within a preset range; and
removing one or more molecules having the molecular weight out of the preset range from the chemical reaction data.

16. The method of claim 15, wherein:
the obtaining of the latent space further comprises:
selecting a preset number of samples to apply at an initial reaction stage;
generating the chemical reaction data corresponding to the preset number of samples; and
generating the chemical reaction learning information with a preset number of single reactions using the chemical reaction data.

17. The method of claim 16, wherein the preset number of single reactions is preset to three.

18. The method of claim 15, wherein the preset range for the molecular weight of the molecules is preset to be greater than 100 g/mol (gram/mole) and less than 300 g/mol.

19. The method of claim 14, wherein the sampling of the similar molecules and the generating of candidate molecules comprise:
combining a first vector in the latent space and a second vector in the embedding space with a start token;
searching for nearest-neighbor (NN) molecules in a space generated by combining of the first and second vectors with the start token;
determining a nearest-neighbor molecule as a starting molecule of chemical reaction; and
updating a reaction sequence.

20. The method of claim 19, wherein the searching for the nearest-neighbor (NN) molecules comprises:
calculating a first Hamming distance between a predictive molecule predicted using a decoder and a predefined starting molecule; and
calculating a second Hamming distance between the determined starting molecule and a seed molecule.
